(19) United States
(12) Patent Application Publication
(10) Pub. No.: US 2025/0152098 A1
(43) Pub. Date: May 15, 2025

(54) Body Temperature-Indicating Clothing Device

(71) Applicant: Cynthia Lee Hirsch, Lakeway, TX (US)

(72) Inventor: Cynthia Lee Hirsch, Lakeway, TX (US)

(21) Appl. No.: 18/401,899

(22) Filed: Jan. 2, 2024

Related U.S. Application Data
(60) Provisional application No. 63/598,712, filed on Nov. 14, 2023.

(51) Int. Cl. A61B 5/00; A61B 5/01

(52) U.S. Cl. CPC A61B 5/6804; A61B 5/01; A61B 5/6803; A61B 5/742; A61B 2560/0214; A61B 2562/0271

(57) ABSTRACT

A body temperature-indicating clothing device is provided. The device is comprised of a piece of clothing comprised of at least one sensor that measures the body temperature of the user. The temperature is then communicated on the clothing via a display or via a color change in the material of the clothing to alert a wearer to their current body temperature.

### CROSS-REFERENCE TO RELATED APPLICATION

The present application claims priority to, and the benefit of, U.S. Provisional Application No. 63/598,712, which was filed on Nov. 14, 2023, and is incorporated herein by reference in its entirety.

### FIELD OF THE INVENTION

The present invention relates generally to the field of clothing. More specifically, the present invention relates to a body temperature-indicating clothing device. The device measures the body temperature of the wearer and displays said temperature to the wearer in a plurality of mechanisms on the device itself. Accordingly, the present disclosure makes specific reference thereto. Nonetheless, it is to be appreciated that aspects of the present invention are also equally applicable to other like applications, devices, and methods of manufacture.

### BACKGROUND

When spending time outdoors in warm weather, individuals may often be oblivious to the changes in their body temperature. As a result, prolonged sun exposure and high temperatures can lead to a variety of heat-related health issues, such as heat stroke, exhaustion, dehydration, and other ailments that might necessitate urgent medical attention. In many cases, people engrossed in outdoor activities may underestimate the intensity of the heat, increasing their risk of overheating. Without a conscious awareness of their rising body temperature, individuals may fail to recognize the critical signs that necessitate a pause in their activities. This oversight can be particularly risky for vulnerable groups such as the elderly, young children, and those with pre-existing health conditions.

Therefore, there exists a long-felt need in the art for a device that helps prevent a user from overheating. There also exists a long-felt need in the art for a body temperature-indicating clothing device. More specifically, there exists a long-felt need in the art for a body temperature-indicating clothing device that records and communicates a wearer's body temperature to the wearer in a visual manner.

The subject matter disclosed and claimed herein, in one embodiment thereof, comprises a body temperature-indicating clothing device. The device is comprised of a piece of clothing comprised of at least one sensor that measures the body temperature of the user. The temperature is then communicated on the clothing via a display or via a color change in the material of the clothing to alert a wearer to their current body temperature.

In this manner, the body temperature-indicating clothing device of the present invention accomplishes all the forgoing objectives and provides a device that helps prevent a user from overheating. There also exists a long-felt need in the art for a body temperature-indicating clothing device. More specifically, there exists a long-felt need in the art for a body temperature-indicating clothing device that records and communicates a wearer's body temperature to the wearer in a visual manner.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a body temperature-indicating clothing device comprised of wearable clothing that measures the body temperature of the wearer of the clothing. The clothing may then display and/or indicate the body temperature of the user on the clothing. In different embodiments, the clothing may be any clothing type such as but not limited to shirts, pants, shorts, hats, glasses, bracelets, socks, shoes, jackets, coats, sweaters, dresses, skirts, ties, scarves, belts, watches, rings, earrings, gloves, tights, leggings, vests, swimwear, sleepwear, underwear, boots, sandals, hoodies, cardigans, etc.

The clothing is comprised of at least one sensor that is preferably a temperature sensor that detects the body temperature of the wearer of the clothing. The sensor is preferably positioned inside the clothing such that the sensor contacts the skin of the wearer in order to accurately measure the body temperature of the wearer. In one embodiment, the clothing is also comprised of at least one electronic display that displays a temperature reading from the sensor in Fahrenheit, Celsius, etc. The clothing may be used for individual use, or for team sport uniforms.

In another embodiment, the clothing is made from a thermochromic material that changes colors based on the body temperature of a user. By way of example, the clothing material may be a first color when a wearer's body temperature is within a first temperature range. Then, the material color may change to a second color when a wearer's body temperature is within a second temperature range that is higher or lower in temperature than the first temperature range. In different embodiments the material of the clothing may change any number of colors in response to any number of body temperature ranges of any interval.

The present invention is also comprised of a method of using the device. First, a device is provided comprised of clothing comprised of at least one body temperature sensor and at least one display. Then, a user can wear the clothing. Next, a user can observe their body temperature as indicated by the sensor and as shown on the display. After observing their body temperature, the user can take measures to cool their body temperature such as but not limited to drinking water, moving into shade, moving into air conditioning, etc.

The present invention is also comprised of a second method of using the device. First, a device is provided comprised of clothing comprised of a thermochromic material that changes from at least a first color to a second color when the wearer's body temperature changes from a first temperature range to a second temperature range. Then, a user can wear the clothing. Next, a user can observe their body temperature as indicated by the color of the clothing. After observing their body temperature, the user can take measures to cool their body temperature such as but not limited to drinking water, moving into shade, moving into air conditioning, etc.

Accordingly, the body temperature-indicating clothing device of the present invention is particularly advantageous as it provides a device that helps prevent a user from overheating. To do so, the device records and communicates a wearer's body temperature to the wearer in a visual manner. In this manner, the body temperature-indicating clothing device provides a solution to prevent a wearer of the device from overheating by making said wearer easily aware of their body temperature.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a body temperature-indicating clothing device of the present invention in accordance with the disclosed architecture;

FIG. 2 illustrates a perspective view of one potential embodiment of a body temperature-indicating clothing device of the present invention in accordance with the disclosed architecture;

FIG. 3 illustrates a flowchart of a method of using one potential embodiment of a body temperature-indicating clothing device of the present invention in accordance with the disclosed architecture;

FIG. 4 illustrates a flowchart of a method of using one potential embodiment of a body temperature-indicating clothing device of the present invention in accordance with the disclosed architecture; and

FIG. 5 illustrates a flowchart of a method of using one potential embodiment of a body temperature-indicating clothing device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a device that helps prevent a user from overheating. There also exists a long-felt need in the art for a body temperature-indicating clothing device. More specifically, there exists a long-felt need in the art for a body temperature-indicating clothing device that records and communicates a wearer's body temperature to the wearer in a visual manner.

The present invention, in one exemplary embodiment, is comprised of a body temperature-indicating clothing device. The device is comprised of wearable clothing that measures the body temperature of the wearer of the clothing. The clothing may then display and/or indicate the body temperature of the user on the clothing. In different embodiments, the clothing may be any clothing type such as but not limited to shirts, pants, shorts, hats, glasses, bracelets, socks, shoes, jackets, coats, sweaters, dresses, skirts, ties, scarves, belts, watches, rings, earrings, gloves, tights, leggings, vests, swimwear, sleepwear, underwear, boots, sandals, hoodies, cardigans, etc.

The clothing is comprised of at least one sensor that is preferably a temperature sensor that detects the body temperature of the wearer of the clothing. The sensor is preferably positioned inside the clothing such that the sensor contacts the skin of the wearer in order to accurately measure the body temperature of the wearer. In one embodiment, the clothing is also comprised of at least one electronic display that displays a temperature reading from the sensor in Fahrenheit, Celsius, etc.

In another embodiment, the clothing is made from a thermochromic material. During use, the material changes colors based on the body temperature of a user. By way of example, the clothing material may be a first color when a wearer's body temperature is within a first temperature range. Then, the material color may change to a second color when a wearer's body temperature is within a second temperature range that is higher or lower in temperature than the first temperature range. In different embodiments the material of the clothing may change any number of colors in response to any number of body temperature ranges of any interval.

The present invention is also comprised of a method of using the device. First, a device is provided comprised of clothing comprised of at least one body temperature sensor and at least one display. Then, a user can wear the clothing. Next, a user can observe their body temperature as indicated by the sensor and as shown on the display. After observing their body temperature, the user can take measures to cool their body temperature such as but not limited to drinking water, moving into shade, moving into air conditioning, etc.

The present invention is also comprised of a second method of using the device. First, a device is provided comprised of clothing comprised of a thermochromic material that changes from at least a first color to a second color when the wearer's body temperature changes from a first temperature range to a second temperature range. Then, a user can wear the clothing. Next, a user can observe their body temperature as indicated by the color of the clothing. After observing their body temperature, the user can take measures to cool their body temperature such as but not limited to drinking water, moving into shade, moving into air conditioning, etc.

Accordingly, the body temperature-indicating clothing device of the present invention is particularly advantageous as it provides a device that helps prevent a user from overheating. To do so, the device records and communicates a wearer's body temperature to the wearer in a visual manner. In this manner, the body temperature-indicating clothing device provides a solution to prevent a wearer of the device from overheating by making said wearer easily aware of their body temperature.

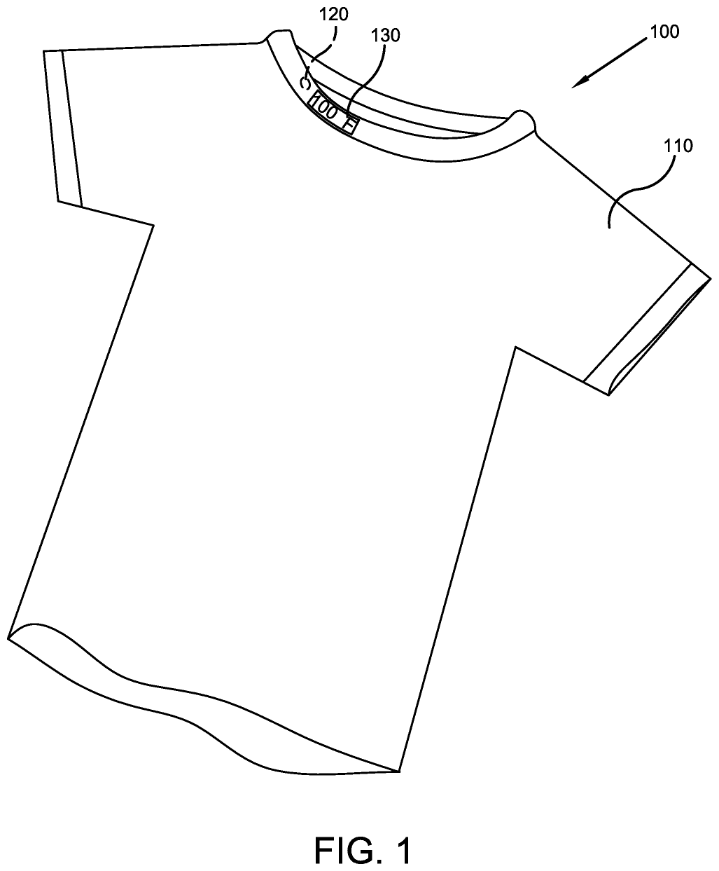

Referring initially to the drawings, FIG. 1 illustrates a perspective view of one potential embodiment of a body temperature-indicating clothing device 100 of the present invention in accordance with the disclosed architecture. The device 100 is comprised of a wearable clothing 110 that measures the body temperature of the wearer of the clothing 110. The clothing 110 may then display and/or indicate the body temperature of the user on the clothing 110.

In different embodiments, the clothing 110 may be any clothing type such as but not limited to shirts, pants, shorts, hats, glasses, bracelets, socks, shoes, jackets, coats, sweaters, dresses, skirts, ties, scarves, belts, watches, rings, earrings, gloves, tights, leggings, vests, swimwear, sleepwear, underwear, boots, sandals, hoodies, cardigans, etc.

The clothing 110 is comprised of at least one sensor 120. The sensor 120 is preferably a temperature sensor that detects the body temperature of the wearer of the clothing 110. The sensor 120 may be positioned anywhere on the clothing 110 but is preferably positioned inside the clothing such that the sensor 120 contacts the skin of the wearer in order to accurately measure the body temperature of the wearer.

In one embodiment, the clothing 110 is also comprised of at least one electronic display 130 that displays a temperature reading from the sensor 120 in Fahrenheit, Celsius, etc. In this embodiment, the display 130 and sensor 120 are powered by at least one battery 150. The battery 150 may be removable or non-removable from the display 130 and/or sensor 120 in different embodiments. The battery 150 may also be rechargeable or non-rechargeable. In a rechargeable and non-removable embodiment, the battery 150 may be comprised of at least one charging port 152 such as but not limited to a USB port.

In another embodiment, the clothing 110 is made from a thermochromic material. This embodiment has no sensor 120 or display 130. Instead, in this embodiment the clothing 110 material changes colors based on the body temperature of a user. By way of example, the clothing 110 material may be a first color when a wearer's body temperature is within a first temperature range. Then, the material color may change to a second color when a wearer's body temperature is within a second temperature range that is higher or lower in temperature than the first temperature range. In different embodiments the material of the clothing 110 may change any number of colors in response to any number of body temperature ranges of any interval.

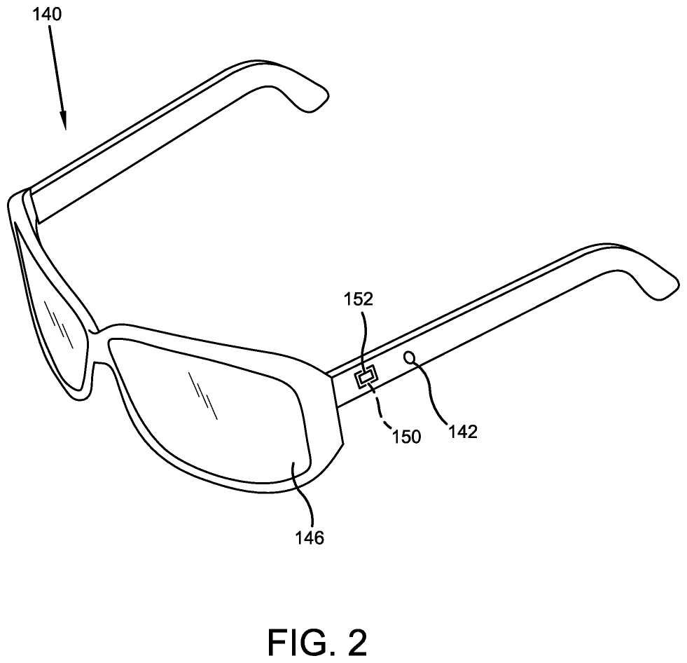

In one embodiment, the clothing 110 is comprised of a pair of glasses 140, as seen in FIG. 2. In this embodiment, the glasses 140 are comprised of at least one sensor 120. Body temperature recorded by the sensor 120 may then be communicated to a user via at least one indicator light 142 on the glasses. The indicator light 142 may illuminate a first color when a wearer's body temperature is within a first temperature range. Then, the light 142 may change to a second color when a wearer's body temperature is within a second temperature range that is higher or lower in temperature than the first temperature range. In different embodiments the light 142 may change any number of colors in response to any number of body temperature ranges of any interval. In a further embodiment, the temperature recorded by the sensor 120 can be displayed on the lens 146.

In this embodiment, the glasses 140 are also comprised of at least one lens 146. The color of the lens 146 may change color in response to the body temperature detected by the sensor 120. For example, the lens 146 may be tinted a first color when a wearer's body temperature is within a first temperature range. Then, the lens 146 may change to a second color when a wearer's body temperature is within a second temperature range that is higher or lower in temperature than the first temperature range. In different embodiments the lens 146 may change any number of colors in response to any number of body temperature ranges of any interval.

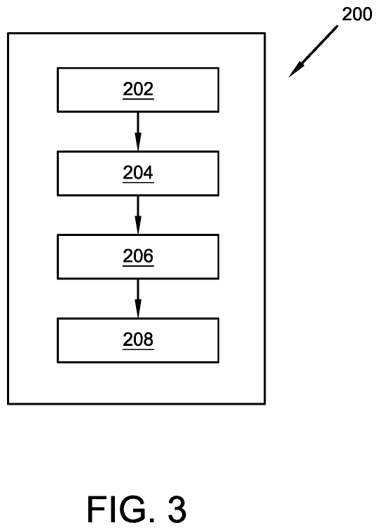

The present invention is also comprised of a method of using 200 the device 100, as seen in FIG. 3. First, a device 100 is provided comprised of a clothing 110 comprised of at least one body temperature sensor 120 and at least one display 130 [Step 202]. Then, a user can wear the clothing 110 [Step 204]. Next, a user can observe their body temperature as indicated by the sensor 120 and as shown on the display 130 [Step 206]. After observing their body temperature, the user can take measures to cool their body temperature such as but not limited to drinking water, moving into shade, moving into air conditioning, etc. [Step 208].

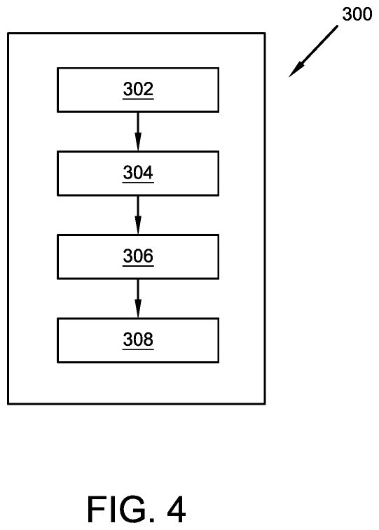

The present invention is also comprised of a second method of using 300 the device 100, as seen in FIG. 4. First, a device 100 is provided comprised of a clothing 110 comprised of a thermochromic material that changes from at least a first color to a second color when the wearer's body temperature changes from a first temperature range to a second temperature range [Step 302]. Then, a user can wear the clothing 110 [Step 304]. Next, a user can observe their body temperature as indicated by the color of the clothing 110 [Step 306]. After observing their body temperature, the user can take measures to cool their body temperature such as but not limited to drinking water, moving into shade, moving into air conditioning, etc. [Step 308].

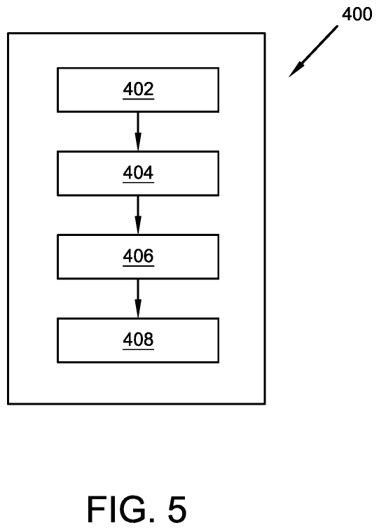

The present invention is also comprised of a third method of using 400 the device 100, as seen in FIG. 5. First, a device 100 is provided comprised of a clothing 110 in the form of a pair of glasses comprised of at least one indicator light 142, at least one sensor 120, and at least one lens 146 which changes from at least a first color to a second color when the wearer's body temperature changes from a first temperature range to a second temperature range [Step 402]. Then, a user can wear the clothing 110 [Step 404]. Next, a user can observe their body temperature as indicated by the indicator light 142 of the clothing 110 [Step 406]. After observing their body temperature, the user can take measures to cool their body temperature such as but not limited to drinking water, moving into shade, moving into air conditioning, etc. [Step 408].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “body temperature-indicating clothing device” and “device” are interchangeable and refer to the body temperature-indicating clothing device 100 of the present invention.

Notwithstanding the forgoing, the body temperature-indicating clothing device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the body temperature-indicating clothing device 100 as shown in the FIGS. are for illustrative purposes only, and that many other sizes and shapes of the body temperature-indicating clothing device 100 are well within the scope of the present disclosure. Although the dimensions of the body temperature-indicating clothing device 100 are important design parameters for user convenience, the body temperature-indicating clothing device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A body temperature-indicating clothing device comprising:
a clothing;
a sensor;
a display; and
a battery.

2. The body temperature-indicating clothing device of claim 1, wherein the sensor is comprised of a body-temperature sensor.

3. The body temperature-indicating clothing device of claim 2, wherein a body-temperature recorded by the sensor is shown on the display.

4. The body temperature-indicating clothing device of claim 1, wherein the clothing is comprised of a shirt, a pair of pants, a pair of shorts, a hat, a pair of glasses, a bracelet, a sock, a shoe, a jacket, a coat, a sweater, a dress, a skirt, a tie, a scarf, a belt, a watch, a ring, an earring, a glove, a pair of tights, a pair of leggings, a vest, a piece of swimwear, a piece of sleepwear, a piece of underwear, a boot, a sandal, a hoodie, or a cardigan.

5. The body temperature-indicating clothing device of claim 1, wherein the battery powers the sensor.

6. The body temperature-indicating clothing device of claim 1, wherein the battery powers the display.

7. The body temperature-indicating clothing device of claim 1, wherein the battery is rechargeable.

8. The body temperature-indicating clothing device of claim 7, wherein the battery is comprised of a charging port.

9. A body temperature-indicating clothing device comprising:
a clothing; and
a thermochromic material, wherein the thermochromic material changes from a first color to a second color based on a first temperature range and a second temperature range of the thermochromic material.

10. The body temperature-indicating clothing device of claim 9, wherein the clothing is comprised of a shirt, a pair of pants, a pair of shorts, a hat, a pair of glasses, a bracelet, a sock, a shoe, a jacket, a coat, a sweater, a dress, a skirt, a tie, a scarf, a belt, a watch, a ring, an earring, a glove, a pair of tights, a pair of leggings, a vest, a piece of swimwear, a piece of sleepwear, a piece of underwear, a boot, a sandal, a hoodie, or a cardigan.

11. A body temperature-indicating clothing device comprising:
a pair of glasses;
a sensor;
an indicator light;
a battery; and
a lens.

12. The body temperature-indicating clothing device of claim 11, wherein the sensor is comprised of a body-temperature sensor.

13. The body temperature-indicating clothing device of claim 12, wherein a body temperature recorded by the sensor causes the indicator light to illuminate a first color or a second color.

14. The body temperature-indicating clothing device of claim 13, wherein the first color correlates to a first temperature range.

15. The body temperature-indicating clothing device of claim 14, wherein the second color correlates to a second temperature range.

16. The body temperature-indicating clothing device of claim 12, wherein a body temperature recorded by the sensor causes the lens to tint a first color or a second color.

17. The body temperature-indicating clothing device of claim 16, wherein the first color correlates to a first temperature range.

18. The body temperature-indicating clothing device of claim 16, wherein the second color correlates to a second temperature range.

19. The body temperature-indicating clothing device of claim 11, wherein the battery powers the indicator light.

20. The body temperature-indicating clothing device of claim 19, wherein the battery powers the sensor.
